(19) United States
(12) Patent Application Publication
(10) Pub. No.: US 2024/0043779 A1
(43) Pub. Date: Feb. 8, 2024

(54) BIOMIMETIC JOINT ON A CHIP

(71) Applicant: SOUTH DAKOTA BOARD OF REGENTS, Pierre, SD (US)

(72) Inventors: SCOTT WOOD, Rapid City, SD (US); RAM SARASWAT, Rapid City, SD (US)

(21) Appl. No.: 18/487,600

(22) Filed: Oct. 16, 2023

Related U.S. Application Data
(63) Continuation of application No. PCT/US2022/021152, filed on Mar. 21, 2022.
(60) Provisional application No. 63/201,248, filed on Apr. 20, 2021.

(51) Int. Cl. C12M 3/06; B01L 3/00; C12M 1/00; C12M 1/12

(52) U.S. Cl. CPC C12M 23/16; B01L 3/502715; C12M 23/22; C12M 25/14

(57) ABSTRACT

A platform for culturing modular, biomimetic compositions such as tissues, cartilage, bone, synovial membrane, is accomplished through the use of a 3D printed platform with cell well, well plate frame with culture and analysis modules, coverglass bottoms for imaging, and cross-talk flow to connect tissue modules for paracrine signaling. Human chondrocytes can be generated and kept in a cell back and expanded to zonal models, osteoarthritis progression models. The use of titanium oxide nanotubes and can produce bone marrow stem cells differentiated toward osteoblasts. The synovial membrane can be modeled by an electrospun mesh, macrophages with an inducible phenotype (quiescent vs. wound repair vs. inflammatory).

### CROSS REFERENCE TO RELATED APPLICATIONS

This application is a By-Pass Continuation of PCT/US2022/021152 filed on Mar. 21, 2022, which application claims priority under 35 U.S.C. § 119 to provisional patent application U.S. Ser. No. 63/201,248 filed Apr. 20, 2021. The provisional patent application is herein incorporated by reference in its entirety, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant Number 2037874 awarded by the National Science Foundation. The government has certain rights in the invention.

### TECHNICAL FIELD

The present disclosure relates to biomimetic joints on a chip and their methods of manufacture and use. Preferably, the biomimetic joints on a chip are modular and suitable for cell cultures and drug screening. Affected industries include at least pharmaceuticals, in vitro drug development, gene therapy, stem cell medicine, tissue engineered scaffolds, elucidation of molecular pathogeneses, and other biomedical industrial applications.

### BACKGROUND OF THE INVENTION

The background description provided herein gives context for the present disclosure. Work of the presently named inventors, as well as aspects of the description that may not otherwise qualify as prior art at the time of filing, are neither expressly nor impliedly admitted as prior art.

Osteoarthritis (“OA”) is a painful disease of the articular joints that is primarily characterized by the degradation of the extracellular matrix (“ECM”) in the articular cartilage. To date, surgical restoration techniques used for cartilage repair do not regenerate hyaline articular cartilage. Although symptoms can improve temporarily after surgical repair, eighty-five percent (85%) of patients progress to failure within seven and a half (7.5) years or less. There are currently no known medical treatments that effectively address the underlying molecular causes of OA. Articular chondrocytes, the cells in the cartilage of our joints, are responsible for the maintenance of cartilage homeostasis between digestion and replacement of old or damaged tissue components. It is well-accepted that a loss of this homeostatic balance is responsible for the development of OA. Current pharmaceutical treatment options are limited to the use of analgesics like non-steroidal anti-inflammatory drugs (“NSAID”) and intra-articular corticosteroid injections to reduce the pain associated with inflammation, which only provides temporary relief and can have negative consequences with long-term use.

For example, with reference to FIG. 1, a limb with a healthy joint 50 is shown on the left, and a limb with a joint affected by OA 60 is shown on the right. Each limb 50, 60 includes muscles 51, synovial bursa 52, tendon 53, bones 54, cartilage 55, synovial membrane 56, and joint capsule 57. Thinned cartilage 58 causes bone ends 59 to rub together, thereby causing the loss of said homeostatic balance and leading to the development of OA.

Animal models have long been the gold standard for understanding the progression of OA. However, they are also associated with concerns of ethical issues regarding the treatment of animals, cost and management issues, anatomical differences of cartilage in animals compared to humans, and age variations of animal species at the time of testing.

Due to the problems associated with animal models, chondrocytes have been studied in vitro using either standard two-dimensional (“2D”) or any number of three-dimensional (“3D”) cell culture techniques. Two-dimensional cell culture techniques are particularly unsuitable for articular chondrocytes. In vivo, articular chondrocyte morphology is generally spheroidal throughout most of the cartilage, and this spheroidal morphology is widely considered to be the canonical morphology of chondrocytes for in vitro studies. Under standard 2D culture conditions, however, chondrocytes tend to develop an artificially induced fibroblastic phenotype after expansion or more than approximately 10 days in culture, which is known to alter their behavior.

Three-dimensional scaffolds have shown promise for promotion of phenotype maintenance of articular chondrocytes and for chondrogenesis of mesenchymal stem cells (“MSCs”), however, although the past decade has realized significant progress in the development of many types of three-dimensional cell culture systems, these techniques are all inherently limited in their utility by restricted oxygen diffusion, restricted and non-uniform penetration of both small molecule and macromolecule treatment agents, and limited optical penetration depth.

Platforms for the growth of cell cultures and testing have been studied. Some platforms have been prepared by others in order to perform pre-clinical research of potential drug therapies in an effort to test toxicity and efficacy. There have been a small number of research groups who have disclosed designs for either cartilage-on-a-chip or a joint-on-a-chip. These were developed based on traditional three-dimensional culture techniques, and suffer from a number of limitations including, diffusion gradient and optical limitations.

Articular chondrocytes a cell type that is very difficult to work with for in vitro studies. In part because of this, no drugs to treat OA have ever successfully completed clinical trials to receive regulatory approval. Not only do primary chondrocytes de-differentiate rapidly (within approximately ten days) when cultured using standard cell culture techniques but attempts to address this problem by developing immortalized chondrocyte cell lines known in the art have failed to adequately match the physiological phenotype of their primary cell counterparts.

Three-dimensional cell culture techniques can enhance phenotypic maintenance of these cells, but these methods tend to severely limit the number of compatible analytical techniques—especially those capable of observing sensitive post-translational modifications of proteins that are key to understanding the molecular mechanisms of cell behavior. The pharmaceutical industry needs access to a system capable of modeling the complexity of the human joint for early phase in vitro drug discovery studies. A system capable of overcoming these technical challenges could rapidly increase progress toward developing more effective treatments for many joint diseases—especially OA.

Thus, there exists a need in the art for improved platforms for the growth of cell cultures.

### SUMMARY OF THE INVENTION

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present invention to improve on or overcome the deficiencies in the art.

It is a further object, feature, and/or advantage of the present invention to provide a modular biomimetic joint-on-a-chip. For example, the biomimetic joint-on-a-chip can be tailored for use with desired cell cultures.

It is still yet a further object, feature, and/or advantage of the present invention to develop and study the effects of drugs for the treatment of joint diseases.

It is still yet a further object, feature, and/or advantage of the present invention to model the joint as an organ during early preclinical studies.

It is still yet a further object, feature, and/or advantage of the present invention to replicate the structure of human articular cartilage.

It is still yet a further object, feature, and/or advantage of the present invention to enabling the modeling of multiple OA pathogenesis pathways.

It is still yet a further object, feature, and/or advantage of the present invention to more easily allow for real-time measurements and imaging of synthetic cells.

It is still yet a further object, feature, and/or advantage of the present invention to design a modular fluidic system offering a robust, high-quality, repeatable data to improve clinical translation of their early preclinical data. Pharmaceutical scientists can use said design to receive regulatory approval for first-ever drugs to halt, prevent, or heal damage due to OA.

It is still yet a further object, feature, and/or advantage of the present invention to enable paracrine signaling between discrete cultures of cells from various tissues of the joint.

It is still yet a further object, feature, and/or advantage of the present invention to develop a biobank of cells from many human donors belonging to a wide variety of healthy and at-risk groups and to replicate the clinical variability present within the population in a repeatable and predictable manner.

It is still yet a further object, feature, and/or advantage of the present invention to prioritize physical cues rather than biochemical cues to keep cells in the system behaving similarly to the way the cells behave in the body. This helps decrease experimental variability. In greater particularity, topographical cues can be used with relative ease to investigate delicate cell signaling mechanisms. Such use of topographical cues over the use of growth factors to drive differentiation can be very beneficial to regulate and maintain physiological phenotypes of those cells, thus minimizing off-target effects.

It is still yet a further object, feature, and/or advantage of the present invention to incorporate electrospun or cast fibers (including, but not limited to, fibers, microfibers, nanofibers, or mixtures thereof) into biomimetic compositions. Electrospun and/or cast fibers can be (a) of an appropriate diameter to match ankle cartilage type II collagen fibers, (b) crosslinked fibers using vapor deposition of glutaraldehyde to prevent them from dissolving in the aqueous environment required for cell culture, and (c) embedded fibers within agarose.

It is still yet a further object, feature, and/or advantage of the present invention to align fibers, identify workable materials (e.g., those that cure slowly enough to pattern), and/or prevent anoikis (massive cell death due to lack of adhesion) during seeding.

It is still yet a further object, feature, and/or advantage of the present invention to functionalize well surfaces. For example, covalent crosslinking methods can be used to adhere extracellular matrix (ECM) proteins to well surfaces. A wide variety of physiologically relevant materials may be incorporated into the hydrogel or used to functionalize well surfaces, including, without limitation, hyaluronic acid-, chondroitin sulfate-, collagen II-derived materials, or polydopamine (“PDA”).

It is still yet a further object, feature, and/or advantage of the present invention to incorporate physiologically relevant materials into a hydrogel and/or more physiologically representative distributions of well geometries, spacings, and nanomaterial arrangement.

The improved platforms for the growth of cell cultures disclosed herein can be used in a wide variety of applications. For example, Further applications in the culture of other cell types, including the stem cell market, where it may help those cells to maintain their stemness during expansion and culture prior to experimentation, are made possible with the present invention.

It is preferred biomimetic joint-on-a-chip be safe to make and use, cost effective, and durable. The cost effective nature of the biomimetic joint-on-a-chip shall help lead to commercial success in the in vitro and arthropathy (i.e., joint disease) segments of the preclinical global contract research organization (“CRO”) market. Regarding durability, cells can be co-cultured from bone, cartilage, and synovium for at least twenty-eight (28), and even up to thirty (30) days while maintaining the viability, physiological morphology, and expression of key phenotypic markers for each cell.

Methods can be practiced which facilitate use, manufacture, assembly, maintenance, and repair of biomimetic compositions which accomplish some or all of the previously stated objectives.

The biomimetic joint-on-a-chip can be incorporated into modular fluidic systems which accomplish some or all of the previously stated objectives.

According to some aspects of the present disclosure, a microfluidic system for culturing modular, biomimetic compositions comprises a platform for the growth of cell cultures and synthetic cells mimicking biochemical materials or processes. The platform comprises a first group of non-collinearly arranged barbed fittings at a first end of said platform and a second group of barbed fittings at a second end opposite said first end. The first and second groups of barbed fittings are capable of establishing fluidic connections between said platform and external devices and/or other fluidic systems. The platform further includes a cell well and/or a removeable window plate located adjacent said second group of barbed fittings and a coverglass bottom for imaging.

According to some additional aspects of the present disclosure, a geometry of the cell well is discoid or triangular. Further, the synthetic cells can be spaced and/or geometrically arranged to mimic or create a cell pairing.

In a specific example, the synthetic cells can be are chondrocytes that model, either independently or in co-culture, a superficial zone, a middle zone, and a deep zone of articular cartilage for both well geometry and nanomaterial arrangement. The chondrocytes can be configured to maintain their spheroidal morphology for a time period of at least twenty-eight days. Expression levels of phenotypic marker proteins in the chondrocytes seeded in the cell well can be at least fifty percent greater than for chondrocytes seeded in monolayer on tissue culture-treated polystyrene culture dishes. The phenotypic marker proteins can be further selected form the group consisting of collagen II, aggrecan, Sox-9 (SRY-Box Transcription Factor 9), and decorin. Expression levels of de-differentiation marker proteins can be at least fifty percent lower than for chondrocytes seeded in monolayer on tissue culture-treated polystyrene culture dishes. The de-differentiation marker proteins cam be further selected from the group consisting of Collagen I, Collagen X, and Ki-67.

According to some other aspects of the present disclosure, a modular, biomimetic composition comprises a natural hydrogel micropatterned with a plurality of wells formed using the microfluidic system described above.

According to some additional aspects of the present disclosure, the biomimetic composition comprises an agarose hydrogel, embedded with nanofibers or nanoparticles and/or is well surface functionalized with PDA. The modular, biomimetic composition can be thin film. The nanofibers can comprise a polyvinyl alcohol, collagen, chitin, or a combination thereof.

According to some additional aspects of the present disclosure, the cell well has an average diameter of from about 5 μm to about 50 μm; and wherein the cell well is separated by an inter-well spacing of from about 0.1 μm to about 30 μm.

According to some other aspects of the present disclosure, a method of culturing modular, biomimetic compositions using a microfluidic system comprises allowing biomimetic fluid to pass through the media inputs into a chamber below an upper surface of the platform, wherein a portion of said chamber includes the cell well and/or space encompassed within the removeable window plate; allowing the biomimetic fluid to pass from the chamber to the media outputs; and using physical cues over biochemical cues to keep the synthetic cells to mimic cell behavior in a human body.

According to some additional aspects of the present disclosure, the method further comprises binding the modular, biomimetic compositions to an antigen, and if binding occurs, producing a detectable signal (which can be a color change); clamping coverslips to thruholes and/or protrusions in the removable window plate; sealing with O-ring that fits into annular grooves located on an outer circumferential surface of the removable window plate; and/or removing air bubbles from aqueous solutions inline or downstream in a the mircofluidic system with a bubble trap.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the present invention can be practiced are illustrated and described in detail, wherein like reference characters represent like components throughout the several views. The drawings are presented for exemplary purposes and may not be to scale unless otherwise indicated.

FIG. 1 shows a comparative view showing one limb with a healthy joint and another limb with a joint affected by OA.

FIG. 2 illustrates a micropatterned thin-film nanocomposite biomaterial-based cell culture platform, specifically utilizing a cell well to maintain the physiological phenotype of primary human articular chondrocytes in vitro while minimizing analytical limitations.

FIG. 3 illustrates merged phase contrast/live (appear as green nuclei)/dead (appear white nuclei) image of chondrocytes in a cell well.

FIG. 4 graphs a comparative view of the compressive modulus of the cell well 106 and cartilage PCM. Nanofibers embedded in the cell well are of similar distribution to ankle cartilage type II collagen fibers.

FIG. 5 graphs a comparative view of the compressive modulus of the cell well 106 and cartilage PCM. The mechanical stiffness closely matches the pericellular matrix (mean±SD).

FIG. 6 graphs a comparative view showing physiological morphology (indicated with aspect ratio) is maintained by the cell well in long-term (days in) culture (mean±SD).

FIG. 7 is a schematic view illustrating various interconnected designs for modular culture chips that can be integrated with various cell culture platforms on glass coverslips.

FIG. 8 renders a perspective view of a first exemplary embodiment of a platform for growth of cell cultures.

FIG. 9 renders a perspective view of a second embodiment of a platform for growth of cell cultures.

FIG. 10 shows an exploded, perspective view of the platform shown in FIG. 9.

FIG. 11 shows a detailed, perspective view of a window plate shown in FIGS. 9-10.

FIG. 12 shows a detailed illustrative view of a thin cell culture scaffold.

FIG. 13 shows an environmental view of a modular fluidic system utilizing the biomimetic joint-on-a-chip partnered with an automated, multiplexed, time-resolved enzyme-linked immunosorbent assay (“ELISA”) system for quantification of secreted enzymes and growth factors.

FIG. 14 charts a layout for a full “knee-on-a-chip” design, according to some aspects of the present disclosure.

An artisan of ordinary skill in the art need not view, within isolated figure(s), the near infinite number of distinct permutations of features described in the following detailed description to facilitate an understanding of the present invention.

### DETAILED DESCRIPTION OF THE INVENTION

The present disclosure is not to be limited to that described herein. Mechanical, electrical, chemical, procedural, and/or other changes can be made without departing from the spirit and scope of the present invention. No features shown or described are essential to permit basic operation of the present invention unless otherwise indicated.

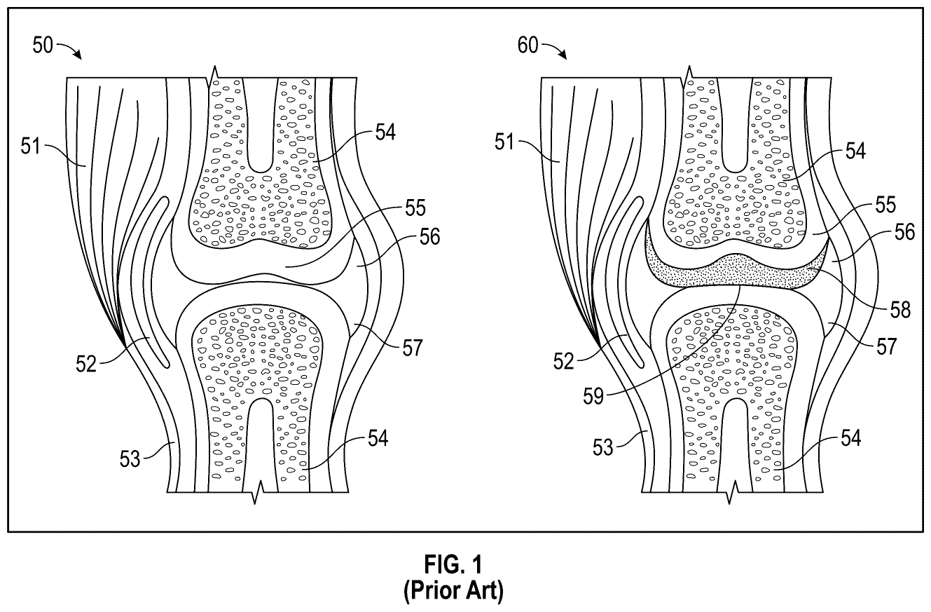

Referring now to the figures, FIG. 1 shows a micropatterned thin-film nanocomposite biomaterial-based cell culture platform 100 based on a “cell well” design. The biomimetic joint-on-a-chip, a type of biomimetic composition, utilizes the unique in vitro cell culture platform 100. The biomimetic joint-on-a-chip dramatically improves control over the differentiation of chondrocytes 102.

Chondrocytes 102 are a notoriously difficult to work with cell type. Chondrocytes 102 maintain their spheroidal morphology over at least about 28 days. The expression levels of phenotypic marker proteins collagen II, aggrecan, Sox-9 (SRY-Box Transcription Factor 9), and decorin in chondrocytes seeded in a cell well will be at least about 50% greater than for chondrocytes seeded in monolayer on tissue culture-treated polystyrene culture dishes. The expression levels of de-differentiation marker proteins Collagen I, Collagen X, and Ki-67 will be at least 50% lower than for chondrocytes seeded in monolayer on tissue culture-treated polystyrene culture dishes.

The culture platform 100 employs a hydrogel 104. In some embodiments, the hydrogel 104 can be a natural, micropatterned hydrogel 104. Preferred micropatterned hydrogels 104 include, but are not limited to, those described in U.S. Pre-Grant Publication (“PG Pub.”) No. 2020/0318050 A1, which is incorporated herein in its entirety. The micropatterned hydrogels 104 have the ability to employ particular substrate geometry(ies) to control chondrocyte differentiation. In a preferred embodiment, the micropatterned hydrogel 104 employed is agarose hydrogels (5% w/v).

Chondrocytes without the addition of exogenous growth factors can have unpredictable side effects. The biomimetic composition can thus be suitable for in vitro drug development, gene therapy, stem cell medicine, tissue engineered scaffolds, elucidation of molecular pathogeneses, and other biomedical applications.

The micropatterned hydrogels 104 can be the component used to make the biomimetic joint-on-a-chip modular. The modularity of the biomimetic joint-on-a-chip allows pharmaceutical scientists to develop drugs for the treatment of joint diseases by providing the ability to model the joint as an organ during early preclinical studies. The modular joint-on-a-chip design is different from the limited number of others in the field based on its use of topographical cues rather than growth factors to drive differentiation. The modular joint-on-a-chip design can be used with relative ease to investigate delicate cell signaling mechanisms.

The micropatterned network of open wells 106 is sized to precisely fit individual cells. This is particularly suitable for chondrocytes 102, which, unlike many other cell types, do not rely on cell-to-cell contact for survival within the body. By basing the biomimetic compositions on an open-well system sized to fit individual cells, each cell is given a three-dimensional “living space” (e.g., well 106) without restricting diffusion of oxygen, other nutrients, or treatments.

The cell well 106 can be designed and manufactured as follows. In some embodiments, the distance between any two consecutive wells varied from two to fifteen micrometers (2 μm to 15 μm). Micropatterned silicon wafers can be obtained and standard contact lithography techniques utilized to generate PDMS cell well stamps. PDMS stamps, can then were sterilized in an autoclave. In a non-limiting example, such sterilization can occur by warming the autoclave to one-hundred twenty-one degrees Celsius (121° C.) for twenty-three (23) minutes. Containment chambers of the cell well 106 can be microfabricated with fifteen micrometer (15 μm)-tall walls, in which the cell well casting process occurs. The walls are constructed to be slightly taller than the hemispheroids in the stamps to provide room for several microns of material to separate the basal surface of the cells from the underlying cover glass without adding excessive bulk that can confound imaging experiments conducted on standard inverted microscopes.

A PVA solution can be obtained by electrospinning PVA nanofibers using an injection rate of one hundred (100) μL/h and an electric potential of five (5) kV. The electrospun nanofibers can then be crosslinked under via glutaraldehyde vapors for forty-eight (48) hours in a vacuum desiccator. In this way, it is possible to consistently produce fibers with diameters closely matching those of ankle articular cartilage. After crosslinking, fibers may be manually chopped to reduce length for use in the nanocomposite casting process.

To cast cell wells 106, molten agarose solution can be mixed with finely chopped crosslinked PVA nanofibers, poured into a containment chamber, and the composite molten solution stamped with a PDMS stamp at four degrees Celsius (4° C.) for six (6) minutes. The stamp can then be removed, revealing the bare cell well 106. Cell wells 106 can then be immediately hydrated with a PBS-lx solution, UV sterilized for thirty (30) minutes, and coated with ten (10) μg/ml each of purified human plasma fibronectin and human placenta collagen type VI for thirty (30) minutes at thirty-seven degrees Celsius (37° C.). For polydopamine (PDA)-functionalized samples, agarose was coated with two (2) mg/mL dopamine-HCl at room temperature followed by coating with twenty-five (25) μg/mL fibronectin for twenty-four (24) hours at thirty-seven degrees Celsius (37° C.).

Alternatively, the cell wells 106 can be coated with a PCM coating 110.

A Keyence VK-X250 optical profilometer can be used to measure the dimensions of cell well features (N=10). To mitigate shrinkage effects in the cell well 106 due to the fact that the gelation mechanism of agarose is solely based on the physical hydrogen-bond networks, and to ensure the fidelity of collected data, cell wells 106 for these measurements can be made out of PVA. PVA can be made by a freeze-thaw method, and frozen samples were able to be utilized to minimize the loss of feature height due to hydrogel drying compared to cells wells 106 made of agarose.

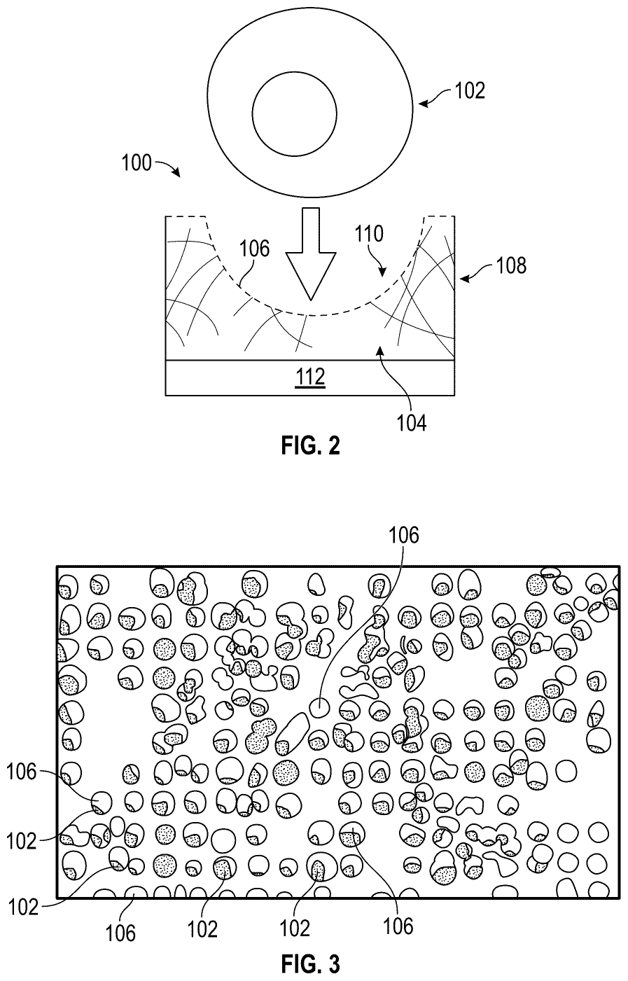

FIG. 2 further shows a unique micropatterned nanocomposite cell culture platform which consists of a thin film with micropatterned with embedded nanofibers 108. The hydrogel substrate 104 that fits a single cell within each well 106 and facilitates high throughput fluorescence imaging of chondrocytes 102.

The biomimetic compositions 100 are able to facilitate those experiments for chondrocytes 102 in a way that also enables the maintenance of their natural phenotype, thereby increasing the translational potential of those experiments over existing technologies. A similar approach can be taken in designing the remaining scaffolds for the joint-on-a-chip, constructing the biomimetic compositions 100 using thin film-based strategies, minimizing diffusion limitations, and relying upon topological cues rather than growth factors to regulate phenotype wherever possible.

Beneficially, the well design for the hydrogel 104 can be varied to incorporate more physiologically representative distributions of (a) well geometries and spacings and (b) arrangement of nanomaterials. The use of varied geometries for the substrate geometry(ies) 106 and varied spacings may include, without limitation, an arrangement mimicking/creating cell pairing, discoid geometries, triangular geometries, etc. With respect to articular chondrocytes specifically, the well geometries, spacings, and materials may be configured to model (either independently or in co-culture) the three zones of articular cartilage (superficial zone, middle zone, deep zone), in both well geometry and nanomaterial arrangement. Beyond articular chondrocytes, the hydrogels 104 and methods of making as described herein may apply to any cell type, including without limitation, stem cells, adipose cells, immune cells, and others.

FIGS. 2-3 shows cell field technologies that incorporate a joint-on-a-chip approach. More particularly, an exemplary modular culture chip 100 is shown integrated with various cell culture platforms 100 on glass coverslips 113. The modular culture chip can be constructed using 3D printing technology. Other suitable methods of manufacturing can also be used, depending on the application. The modular culture chip 100 is compatible with the micropatterned hydrogels 104 and other cell culture substrates.

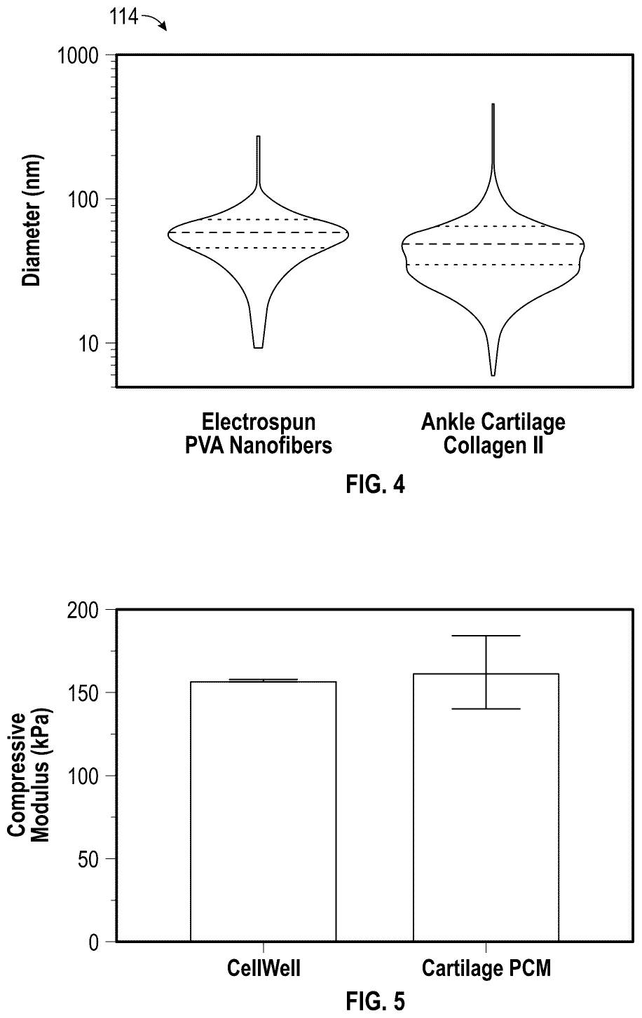

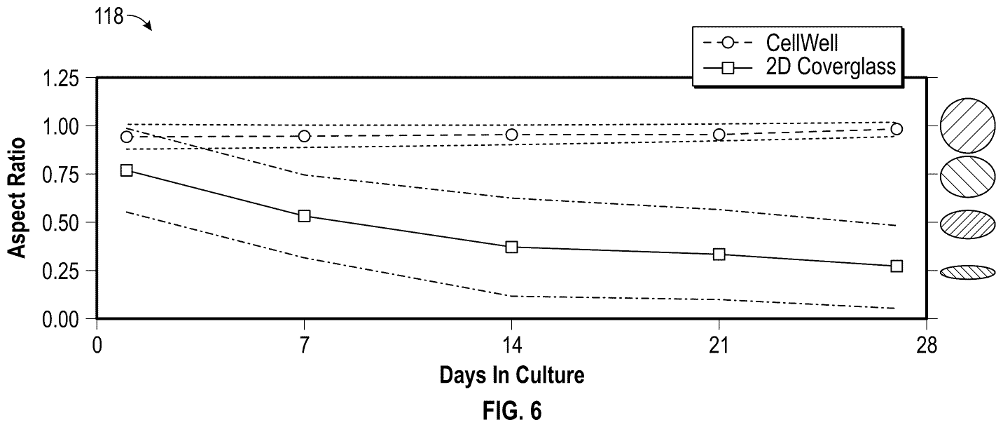

In some embodiments, the substrate composition was chosen to recapitulate the ECM of articular cartilage wherein a hydrogel models cartilage proteoglycans and embedded nanofibers model collagen II fibers. As shown in FIGS. 4-6, the cell wells 106 can be designed such that: (1) their geometries reinforce the canonical spheroidal chondrocyte morphology for each cell 106 (FIG. 6); (2) mechanical stiffness of articular cartilage ECM or the chondrocyte pericellular matrix (PCM) are matched as closely as possible (FIG. 5); (3) the diameters of the embedded nanofiber diameters are matched as closely as possible to those of the native collagen II fibers (FIG. 4); and (4) to be compatible with traditional cell culture and live-cell imaging techniques.

As can be further seen in FIG. 4, the collagen II nanofibers can have a median diameter of fifty nanometers (50 nm) compared to the sixty nanometers (60 nm) median diameter of PVA nanofibers. The PVA nanofibers were found to be within ten nanometers (10 nm) for the median as well as the twenty-fifth (25^{th}) and seventy-fifth (75^{th}) quartiles of the ankle collagen II nanofibers as well, substantiating the use of PVA nanofibers to model the collagen II nanofibers in the cell well 106.

FIG. 6 shows aspect ratio measurements (mean±S.D., n=150 cells) over a period of 4 weeks show strong long-term maintenance of spheroidal morphology by the cell well 106 (p<0.0001 relative to 2D coverglass at each time point).

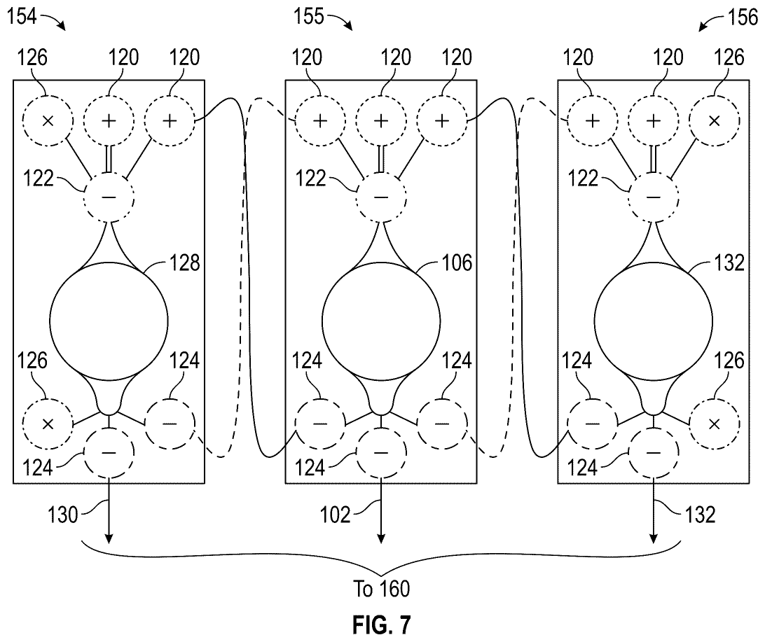

FIG. 7 shows the interconnected nature of modular culture chips that can be integrated with various cell culture platforms 100 on glass coverslips 113. The joint-on-a-chip system includes in vitro models of the articular cartilage 54, underlying bone 55, and the synovial joint capsule 56. The three designs 154, 155, 156 shown left to right, are designed to culture mesenchymal stem cells 130 primary human articular chondrocytes 102, and human THP-1 macrophages 134, respectively. The three designs 154, 155, 156 incorporate titanium dioxide nanotubes (“TiO₂ NTs”) 128, live nuclei within cell wells 106, and electrospun/cast nanofibers 132, respectively. Other types of cells, such as human hFOB 1.19 osteoblasts 191 can also be cultured using similar designs.

The TiO₂ NTs 128 can be transparent. The TiO₂ NTs 128 can be adhered to glass coverslips 113 to establish a method of capturing and quantifying intricate cellular responses in live cells in real-time. Fabrication of transparent TiO₂ NTs 128 can be accomplished by (a) anodization of a thin titanium foil and transferring the foil to a conductive substrate, or application of a thin layer of titanium, via thermal evaporation or RF sputtering, onto glass or fluorine-doped tin oxide (FTO)-coated glass. In some embodiments, the transparent TiO₂ NTs 128 allow for the control over nanotube diameter which can vary.

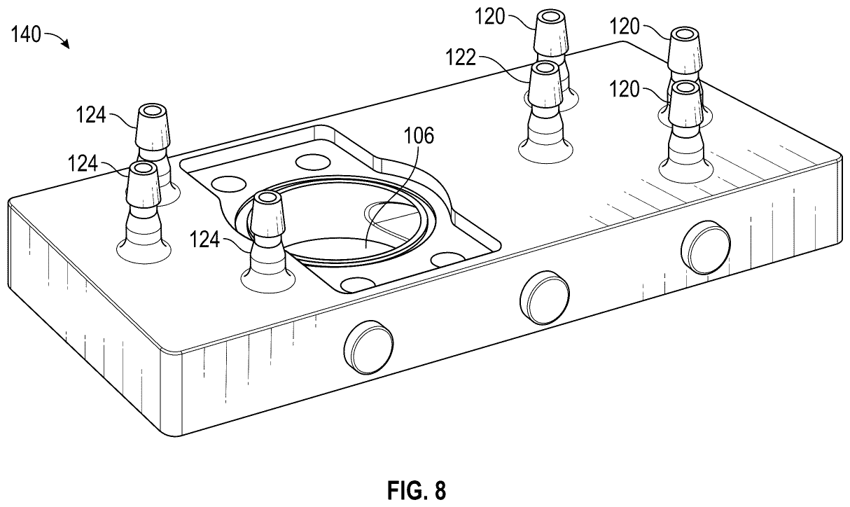

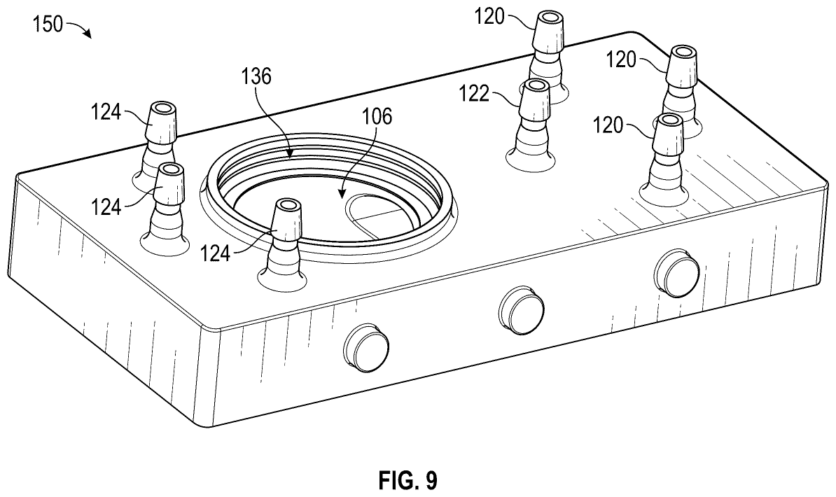

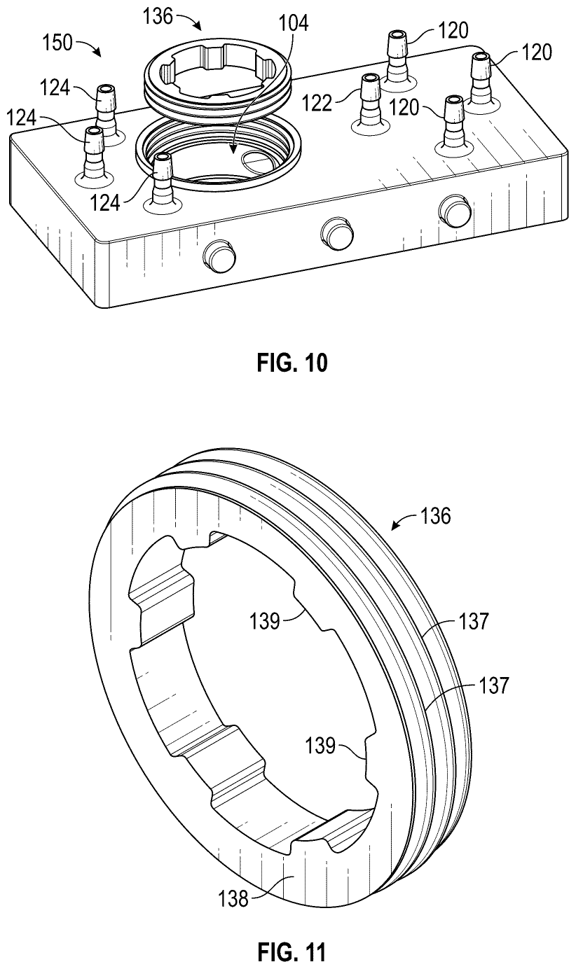

In particular, FIG. 8 is a surface rendering of a computer aided design (“CAD”) file for a first single 3D printed modular culture chip (generically 140, specifically shown as 154, 155, and 156) showing barbed fittings for media input(s) 120, a barbed fitting including a bubble trap 122, and barbed fittings for media output(s) 124. Further aspects of another 3D printed prototype 150 are shown in FIGS. 9-11.

As shown in FIGS. 8-9, the barbed fittings 120, 122, 124 are located at opposite ends of an upper surface of platforms 140, 150. The window plate fitting 136 and/or cell well 106 is located near a second end (adjacent media outputs 124), opposite a first end (adjacent media inputs 120 and bubble trap 122).

Though the embodiments 140, 150 of FIGS. 8-9 show the media inputs 120 and bubble trap 122 arranged at the corners of a diamond toward the first end, and the media outputs 124 shown arranged as the corners of an isosceles triangle toward the second end, it is to be appreciated a greater or lesser number and/or different orientations/arrangements of media inputs and outputs 120, 124 can be employed. Likewise, though the inputs and outputs 120, 124 are shown each having one “barb” (i.e., a sharp projection near the end of an arrow-like item, angled away from the main point so as to make extraction difficult), any number of barbs can be employed to facilitate securement. That said, to reduce mechanical instability and stresses on the system and to create a more robust platform 100, it can be beneficial if the barbed fittings 120, 122 located near the first end and the barbed fittings 124 located near the second end are not collinearly arranged. Moreover, added strength and stability can be achieved where two fittings are equidistantly and oppositely displaced from a central axis of the platform 100 running from the first end to the second end.

The barbed fittings 120, 122, 124 can be quick connect fittings, (i.e., couplings used to provide a fast, make-or-break connection of fluid transfer lines). Operated by hand, the barbed fittings 120, 122, 124 can be pushed together to establish securement. This eliminates the need for threaded or flanged connections, which often require tools. However, it is to be appreciated that traditional fasteners (threads, flanges, magnets, screws, etc.) connections can be used in some embodiments to facilitate securement. The quick connect fittings can be equipped with self-sealing valves or gaskets, such that, upon disconnection, the quick connect fittings automatically contain any fluid in the line.

The open culture window, formed from window plate 136 (a detailed view of which can be seen by way of FIG. 11), includes grooves 137 for an O-ring seal and thruholes for clamps to secure coverslips 113. The O-ring can be a packing or a toric joint, a mechanical gasket in the shape of a torus, or a loop of elastomer with a round cross-section. The O-ring can be designed to be seated in the groove(s) 137 and compressed during assembly between two or more parts, creating a compressed seal at the interface. The O-ring can be used in static applications or in dynamic applications (there is relative motion between the parts of platform(s)/joint(s) 100, 140, 150 and the O-ring). Static applications of O-rings include fluid and/or gas sealing applications in which the O-ring is compressed resulting in zero clearance, the O-ring material is vulcanized solid such that it is impermeable to the biomimetic fluid or gas, and/or the O-ring material is resistant to degradation by the fluid or gas. There wide range of potential biomimetic liquids and gases that must be considered in order to select the ideal material for the O-ring. The selected material for the manufacture of the O-rings is ideally the most inexpensive and easy to manufacture material, so long as the O-rings are still mechanically reliable (e.g., a maximum recommended pressure, seal hardness, and gland clearance of the O-ring seal are safely achieved) and include simple mounting requirements.

Thruholes can extend from an outer circumferential surface of an annular body making up the window plate 136 through to an inner circumferential surface of the annular body. Protrusions 139 located on the inner circumferential surface of the annular body can also facilitate securement of said clamps. The cell culture substrates 112 will be secured at lower radial surface 138 (the bottom) of the open culture window plate 136.

As illustrated in FIGS. 8-10, biomimetic fluid can enter through media inputs 120 toward a chamber that exists below an upper surface of the platform(s) 100, 140, 150. The fluid is then allowed to travel from a first end of the platform to a second end of the platform, near cell well 106 and/or removable window plate 136. After allowing being used to culture cells, such as chondrocytes 102, for a time, said fluids are then allowed to pass through media outlets 126.

Use of a bubble trap 122 can help remove air bubbles from aqueous solutions inline or downstream in a fluidic system. Without the bubble trap 122, the system can experience sudden shear force variations, which changes the compliance of the system, or even blocks small fluid channels. Bubble traps 122 can thus be critical to ensure a safe performance in some embodiments.

Depending on application, an absence 126 of an input/output or non-utilization 126 of any one or more of the barbed fittings can form part of the design 154/155/156. Specifically, the barbed fittings are the media inputs 120, media input containing bubble trap 122, and media outputs 124 that establish fluidic connections to other fluidic systems and components.

A micropatterned hydrogel 104 was employed as the substrate 112 for the cartilage module of design 154. The ‘containment chamber’ system can be combined with an electrospinning apparatus used to generate poly(vinyl alcohol) (PVA) nanofibers for the cell well 106 to generate thin film electrospun meshes of polycaprolactone (PCL) for the synovial membrane substrate 112. Depending on application, PCL can be used in lieu of PVA because of its ease of use with electrospinning and its capability to generate larger diameter nanofibers that reflect the nature of the type I collagen fibers in the synovial membrane more closely than the diameters achieved with PVA for modeling the type II collagen fibers in the cartilage.

The inventors of the present invention have shown the ability to conduct live-cell imaging on a system of TiO₂ NTs. Co-owned U.S. Pat. No. 7,974,853, which is herein incorporated by reference in its entirety, describes further techniques for minimizing nitrous oxide emissions and increasing certainty in generating, quantifying and verifying standardized environmental attributes relating to nitrous oxide. As mentioned above, the TiO₂ NTs 128 can be used as the substrate 112 for the bone component of design 154. While the osteogenic potential of TiO₂ NTs 128 is well established in the literature, there remains some debate regarding the ideal diameter to promote osteogenic differentiation. The source of variability between studies in the literature is likely differences in surface energy and titanium crystallinity due to differences in manufacturing practices between labs. Thus, completion of a twenty-eight (28) day study of human bone-marrow derived mesenchymal stem cells (hBMSCs) on TiO₂ NTs 128 of various diameters has identified which diameter is the most osteogenic using our manufacturing techniques. TiO₂ NTs 128 with the most osteogenic diameter can be used for further research.

The preclinical CRO market can be further broken down into in vivo vs. in vitro segments or be broken down by disease. The overlap between the in vitro and arthropathy (i.e., joint disease) segments provides a broader view of the overall market landscape (e.g., global OA therapeutics market) that shows said market currently comprised primarily of analgesics and dietary supplements sold by pharmaceutical and nutraceutical companies. Yet, these treatments do little-to-nothing to slow or reverse OA. Viscosupplementation therapies are a growing component of this market, but these, by regulatory definition, are also not disease-modifying treatments. By contrast, the global joint replacement market, currently serviced by medical device companies and orthopedic healthcare providers, is much larger. Preclinical contract research services can enable pharmaceutical companies to develop the first-ever disease-modifying osteoarthritis drugs (“DMOADs”), thus leading to restructuring and explosive growth of the OA therapeutics market at the expense of the joint replacement market.

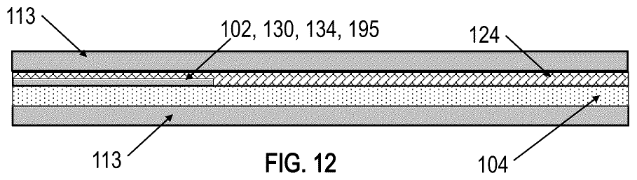

Preferably, the biomimetic composition is thin film, only approximately as thick as the cells (e.g., chondrocytes 102) themselves. By limiting the thickness of the micropatterned hydrogels 104 (only ˜7 μm of material between the cells and the underlying coverglass substrate), the biomimetic compositions are compatible with even the most advanced bioimaging techniques. One such implementation for the substrate 112 is a thin cell culture scaffold as shown in FIG. 12. The thin cell scaffold shown can be a porous scaffold by freeze drying synthetic/natural components. The thin cell culture scaffold shown includes has a thickness that is preferably less than less than 50 um; less than 40 um; less than 30 um; between 10-25 um; most preferably between 15-20 um. The textured, micropatterned hydrogel substrate 104 of the scaffold is shown sandwiched between a hard transparent lid on top (e.g., a glass coverslip 113) and a hard, transparent scaffold substrate (e.g., a glass coverslip 113). Such glass coverslips 113 can have a thickness of approximately 150 um. In some embodiments, the ratio of glass coverslips 113 thickness to substrate thickness is between 3 and 50, more preferably between 5 and 25, and most preferably between 10 and 15. The size of the cell culture media 124 included can heavily vary depending on the application. For example, cell culture media thickness can be between 200 um and 2 cm.

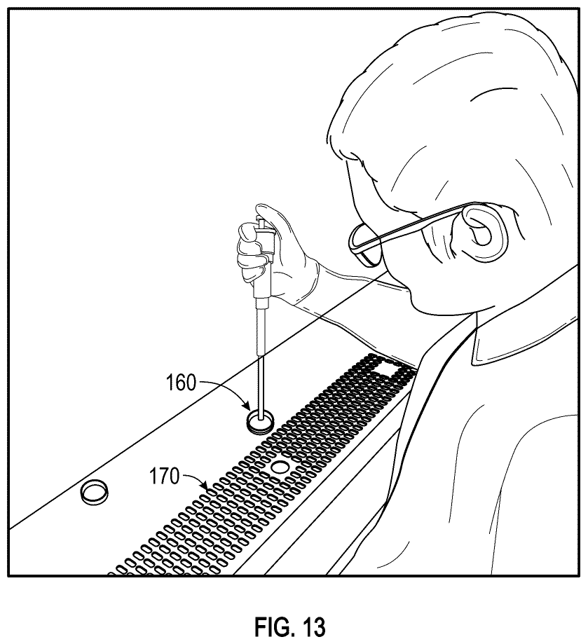

These design features facilitate the easy use of high throughput single cell imaging and analysis techniques, thereby drastically increasing statistical power over conventional batch measurement approaches. Furthermore, as shown in FIG. 13, the modular fluidic system utilizing the biomimetic joint-on-a-chip can be partnered with an automated, multiplexed, time-resolved enzyme-linked immunosorbent assay (“ELISA”) system 170 for quantification of secreted enzymes and growth factors. This allows the modular fluidic system to be capable of efficiently conducting a full in vitro characterization of joint health in response to investigational drugs.

Depending on application, the thickness of the thin film can be characterized by a distance selected from the group consisting of: less than 1 mm, less than about 0.5 mm, less than about 0.2 mm, less than about 150 micrometers, less than about 120 micrometers, less than about 100 micrometers, no more than about 90 micrometers, no more than about 80 micrometers, no more than about 75 micrometers, no more than about 70 micrometers, no more than about 60 micrometers, no more than about 50 micrometers, no more than about 40 micrometers, no more than about 30 micrometers, no more than about 25 micrometers, no more than about 20 micrometers, and no more than about 15 micrometers. Traditional cell culture platforms are often greater than 1 millimeter in thickness. This limits the optical testing that can be performed on the platform. Creating thin platforms has proven difficult in that the three-dimensional nature of an in vitro cell is lost making the platform unsuitable for proper testing. One benefit of the present disclosure is that these biomimetic joints on a chip are thin film, suitable for optical analysis, and retain three-dimensional structure desired for proper analysis and in vitro studies.

In ELISA, antigens from the sample to be tested are attached to a surface. A matching antibody is applied over the surface so it can bind the antigen. This antibody is linked to an enzyme and then any unbound antibodies are removed. A substance containing the enzyme's substrate is added. If binding occurs, the subsequent reaction produces a detectable signal, such as a color change.

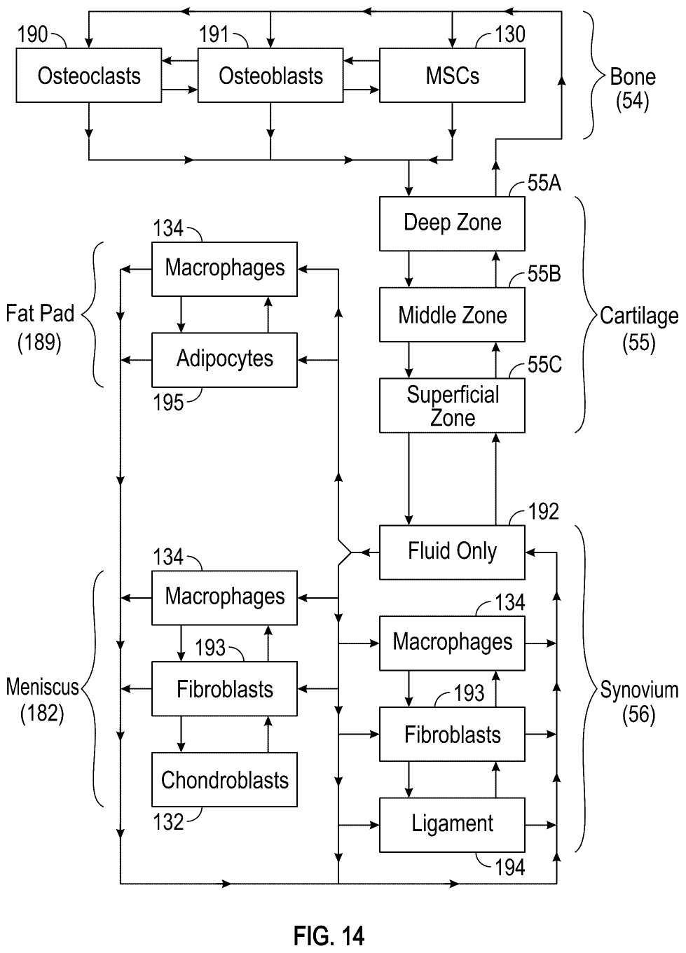

FIG. 14 shows a comprehensive layout for a full “knee-on-a-chip” design 180. The design 180 includes five major zones, which emulate the bone 54, cartilage 55, synovium 56, meniscus 182, and fat pad 189 of the knee. The bone zone includes osteoclasts 190, osteoblasts 191, and mesenchymal stem cells 130, which are illustratively connected to a deep zone 55A (subzone) of the cartilage zone.

The cartilage zone of the microfluidic system for culturing modular, biomimetic compositions for the knee-on-a-chip design 180 models, either independently or in co-culture, a superficial zone 55C, a middle zone 55B, and a deep zone 55A of articular cartilage for both well geometry and nanomaterial arrangement. The superficial zone 55C (subzone of cartilage zone) is illustratively connected to synovium fluid 192 within the synovium zone. The synovium zone also models macrophages 134, fibroblasts 193, and ligament 194, all of which are illustratively connected to each component of the meniscus zone and fat pad zone, such as additional chondroblasts 132, macrophages 134, fibroblasts 193, and adipocytes 195 dedicated to those zones

Referring now to the entirety of the present disclosure, it is to be appreciated the use of the technology of the present disclosure (e.g., cell well 106 and TiO₂ nanotubes 128) increases chances of regulatory success by maximizing reproducibility. By utilizing these cell culture technologies to direct cell behavior, more predictable results than traditional cell culture or small animal studies can be produced. Furthermore, a human donor bio-bank can be built and used to model specific population subsets and total clinical variability. This donor bank, together with the directive behavior mentioned above, enables achievement of a level of reproducibility that has never been available previously.

The use of this technology (e.g., the cell well 106 and TiO₂ nanotubes 128) increases chances of regulatory success by directing/maintaining cell phenotypes without the need for exogenous growth factors. Exogenous growth factors are commonly used to regulate cell phenotypes but can lead to regulatory challenges due to unforeseen off-target effects. This technology utilizes only physical cues to regulate phenotype, and, thus, will decrease regulatory hurdles in the translation of preclinical data into clinical studies.

The use of this technology (e.g., the cell well 106 and TiO₂ nanotubes 128) increases chances of successful translational success to large animal models by at least 10%. For example, this can be achieved by more accurately modeling the physiology of the joints of large animals (including humans) than small animals do.

The use of this technology (e.g., the cell well 106 and TiO₂ nanotubes 128) reduces dependence on small animal models for predicting drug efficacy within a feasible budget.

From the foregoing, it can be seen that the present invention accomplishes at least all of the stated objectives.

### Example Embodiments

The inventions are defined in the claims. However, below is provided a non-exhaustive list of non-limiting embodiments. Any one or more of the features of these embodiments may be combined with any one or more features of another example, embodiment, or aspect described herein.

1. A method of culturing modular, biomimetic compositions comprising:
providing a microfluidic system comprising:
   a platform (100, 140, 150) for the growth of cell cultures, said platform comprising:
   a first group of non-collinearly arranged barbed fittings (120, 122, 124) at a first end of said platform (100, 140, 150);
   a second group of barbed fittings (120, 122, 124) at a second end opposite said first end;
   wherein said first and second groups of barbed fittings (120, 122, 124) are capable of establishing fluidic connections between said platform (100, 140, 150) and external devices and/or other fluidic systems;
   a cell well (106) and/or removeable window plate (136) located adjacent said second group of barbed fittings (120, 122, 124); and
   a transparent bottom substrate (e.g., 112) for imaging;
   synthetic cells (e.g., 102, 130, 134, 195) mimicking biochemical materials or processes
allowing biomimetic fluid (192) to pass through the inputs (120) into a chamber below an upper surface of the platform (100), wherein a portion of said chamber includes the cell well (106) and/or space encompassed within the removeable window plate (136);
allowing the biomimetic fluid (192) to pass from the chamber to the media outputs (124); and
using physical cues rather than biochemical cues to keep the synthetic cells (e.g., 102, 130, 134, 195) to mimic cell behavior in a human body.

2. The method of paragraph 1 further comprising binding the synthetic cells (e.g., 102, 130, 134, 195) to an antigen, and if binding occurs, producing a detectable signal.

3. The method of paragraph 2 wherein the detectable signal is a color change.

4. The method of any one of paragraphs 1-3 further comprising clamping coverslips (113) to thru holes and/or protrusions (139) in the removable window plate (136).

5. The method of any one of paragraphs 1-4 further comprising sealing with O-ring that fits into annular grooves (139) located on an outer circumferential surface of the removable window plate (136).

6. The method of any one of paragraphs 1-5 further comprising removing air bubbles from aqueous solutions inline or downstream in a the mircofluidic system with a bubble trap (122).

7. A microfluidic system for culturing modular, biomimetic compositions comprising:
a platform (100, 140, 150) for the growth of cell cultures, said platform comprising:
   a first group of non-collinearly arranged barbed fittings (120, 122, 124) at a first end of said platform (100, 140, 150);
   a second group of barbed fittings (120, 122, 124) at a second end opposite said first end;
   wherein said first and second groups of barbed fittings (120, 122, 124) are capable of establishing fluidic connections between said platform (100, 140, 150) and external devices and/or other fluidic systems;
   a cell well (106) and/or removeable window plate (136) located adjacent said second group of barbed fittings (120, 122, 124); and
   a transparent bottom substrate (e.g., 112) for imaging;
synthetic cells (e.g., 102, 130, 134, 195) mimicking biochemical materials or processes.

8. The microfluidic system for culturing modular, biomimetic compositions of paragraph 7 wherein a geometry of the cell well (106) is discoid or triangular.

9. The microfluidic system for culturing modular, biomimetic compositions of any one of paragraphs 7-8 wherein the synthetic cells (e.g., 102, 128, 132, 195) are spaced and/or geometrically arranged to mimic or create a cell pairing.

10. The microfluidic system for culturing modular, biomimetic compositions of any one of paragraphs 9-10 wherein the synthetic cells (e.g., 102, 128, 132, 195) are chondrocytes (102) that model, either independently or in co-culture, a superficial zone (55C), a middle zone (55B), and a deep zone (55A) of articular cartilage for both well geometry and nanomaterial arrangement.

11. The microfluidic system for culturing modular, biomimetic compositions of paragraph 10 wherein the chondrocytes (102) are configured to maintain their spheroidal morphology for a time period of at least twenty-eight days.

12. The microfluidic system for culturing modular, biomimetic compositions of paragraph 10 or 11 wherein expression levels of phenotypic marker proteins in the chondrocytes (102) seeded in the cell well (106) are at least fifty percent greater than for chondrocytes (102) seeded in monolayer on tissue culture-treated polystyrene culture dishes.

13. The microfluidic system for culturing modular, biomimetic compositions of paragraph 12 wherein the phenotypic marker proteins are selected form the group consisting of collagen II, aggrecan, Sox-9 (SRY-Box Transcription Factor 9), and decorin.

14. The microfluidic system for culturing modular, biomimetic compositions of paragraph 11 wherein expression levels of de-differentiation marker proteins are at least fifty percent lower than for chondrocytes (102) seeded in monolayer on tissue culture-treated polystyrene culture dishes.

15. The microfluidic system for culturing modular, biomimetic compositions of paragraph 14 wherein the de-differentiation marker proteins are selected from the group consisting of Collagen I, Collagen X, Ki-67, and decorin.

16. The microfluidic system for culturing modular, biomimetic compositions of any one of paragraphs 7-15 wherein the synthetic cells (e.g., 102, 128, 132, 195) are mesenchymal stem cells (130), adipose cells (195), or immune cells.

17. A modular, biomimetic composition comprising:
a natural hydrogel (104) micropatterned with a plurality of wells formed using the microfluidic system for culturing modular, biomimetic compositions of paragraph 1.

18. The modular, biomimetic composition of paragraphs 17 wherein the natural hydrogel (104) is an agarose hydrogel.

19. The modular, biomimetic composition of any one of paragraphs 17-18 wherein the well surface is functionalized with polydopamine (“PDA”).

20. The modular, biomimetic composition of any one of paragraphs 17-19 wherein the modular, biomimetic composition is thin film.

21. The modular, biomimetic composition of any one of paragraphs 17-20 wherein the hydrogel (104) comprises a nanofibers and/or nanoparticles (108) embedded within the hydrogel (104).

22. The modular, biomimetic composition of paragraphs 15 wherein the nanofibers (108) comprises a polyvinyl alcohol, collagen, chitin, or a combination thereof.

23. The modular, biomimetic composition of any one of claims 17-22 wherein the cell well (106) has an average diameter of from about 5 μm to about 50 μm; and wherein the cell well (106) is separated by an inter-well spacing of from about 0.1 μm to about 30 μm.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 50 | limb with healthy joint |
| 51 | muscle |
| 52 | synovial bursa |
| 53 | tendon |
| 54 | bone |
| 55 | cartilage |
| 55A | superficial zone |
| 55B | middle zone |
| 55C | deep zone |
| 56 | synovial membrane |
| 57 | joint capsule |
| 58 | thinned cartilage |
| 59 | bone ends |
| 60 | limb with joint affected by osteoarthritis |
| 100 | culture platform/biomimetic joint-on-a-chip |
| 102 | chondrocyte |
| 104 | hydrogel |
| 106 | well |
| 108 | embedded nanofibers |
| 110 | pericellular matrix coating |
| 112 | substrate |
| 113 | glass coverslips |
| 114 | graph comparing nanofiber diameter |
| 116 | graph comparing material stiffness |
| 118 | graph comparing long-term chondrocyte morphology |
| 120 | media input |
| 122 | bubble trap |
| 124 | media output |
| 126 | absence of/non-utilized input/output |
| 128 | titanium dioxide nanotubes |
| 130 | mesenchymal stem cells |
| 132 | electrospun/cast nanofibers |
| 134 | macrophages |
| 135 | transparent . . . |
| 136 | window plate |
| 137 | circumferential groove |
| 138 | radial surface of annular body |
| 139 | radially arrayed inward protrusions |
| 140 | first exemplary 3D printed platform |
| 150 | second exemplary 3D printed platform |
| 154 | platform configuration for bone cells |
| 155 | platform configuration for cartilage |
| 156 | platform configuration for synovium |
| 160 | analysis chips |
| 170 | enzyme-linked immunosorbent assay system |
| 180 | knee on a chip |
| 182 | meniscus |
| 189 | fat pad |
| 190 | osteoblasts |
| 191 | osteoclasts |
| 192 | fluid |
| 193 | fibroblasts |
| 194 | ligament |
| 195 | adipocytes |

### Glossary

Unless defined otherwise, all technical and scientific terms used above have the same meaning as commonly understood by one of ordinary skill in the art to which embodiments of the present invention pertain.

The terms “a,” “an,” and “the” include both singular and plural referents. The term “or” is synonymous with “and/or” and means any one member or combination of members of a particular list.

The terms “invention” or “present invention” are not intended to refer to any single embodiment of the particular invention but encompass all possible embodiments as described in the specification and the claims.

The term “about” as used herein refer to slight variations in numerical quantities with respect to any quantifiable variable, including, but not limited to, mass, volume, time, temperature, length, density, etc. Inadvertent error can occur, for example, through use of typical measuring techniques or equipment or from differences in the manufacture, source, or purity of components. The term “about” also encompasses amounts that differ due to different equilibrium conditions for a composition resulting from a particular initial mixture. Whether or not modified by the term “about,” the claims include equivalents to the quantities.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The term “actives” or percent actives” or “percent by weight actives” or “actives concentration” are used interchangeably herein and refers to the concentration of those ingredients expressed as a percentage minus inert ingredients such as water or salts.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

A “hydrogel” as used herein refers to a polymeric material which exhibits the ability to swell in water and to retain a significant portion of water within its structure without dissolution. Hydrogels are typically three-dimensional macromolecular networks in water formed from a cross-linked polymer.

The term “nanofiber” as used herein refers to fibers with diameters smaller than of 1.0 micrometer, and generally between 10 nanometers and 1.0 micrometer, such as between 200 nm and 600 nm.

The term “composite nanofibers” as used herein are nanofibers produced from at least two different polymers.

The enzyme-linked immunosorbent assay (“ELISA”) is a plate-based assay technique designed for detecting and quantifying peptides, proteins, antibodies, and hormones. The assay uses a solid-phase type of enzyme immunoassay (EIA) to detect the presence of a ligand (commonly a protein) in a liquid sample using antibodies directed against the protein to be measured.

The “scope” of the present invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

What is claimed is:

1. A method of culturing modular, biomimetic compositions comprising:
providing a microfluidic system comprising:
a platform (100, 140, 150) for the growth of cell cultures, said platform comprising:
a first group of non-collinearly arranged barbed fittings (120, 122, 124) at a first end of said platform (100, 140, 150);
a second group of barbed fittings (120, 122, 124) at a second end opposite said first end;
wherein said first and second groups of barbed fittings (120, 122, 124) are capable of establishing fluidic connections between said platform (100, 140, 150) and external devices and/or other fluidic systems;
a cell well (106) and/or removeable window plate (136) located adjacent said second group of barbed fittings (120, 122, 124); and
a transparent bottom substrate (e.g., 112) for imaging;
synthetic cells (e.g., 102, 130, 134, 195) mimicking biochemical materials or processes
allowing biomimetic fluid (192) to pass through the inputs (120) into a chamber below an upper surface of the platform (100), wherein a portion of said chamber includes the cell well (106) and/or space encompassed within the removeable window plate (136);
allowing the biomimetic fluid (192) to pass from the chamber to the media outputs (124); and
using physical cues rather than biochemical cues to keep the synthetic cells (e.g., 102, 130, 134, 195) to mimic cell behavior in a human body.

2. The method of claim 1 further comprising binding the synthetic cells (e.g., 102, 130, 134, 195) to an antigen, and if binding occurs, producing a detectable signal.

3. The method of claim 2 wherein the detectable signal is a color change.

4. The method of claim 1 further comprising clamping coverslips (113) to thru holes and/or protrusions (139) in the removable window plate (136).

5. The method of claim 1 further comprising sealing with O-ring that fits into annular grooves (139) located on an outer circumferential surface of the removable window plate (136).

6. The method of claim 1 further comprising removing air bubbles from aqueous solutions inline or downstream in a the mircofluidic system with a bubble trap (122).

7. A microfluidic system for culturing modular, biomimetic compositions comprising:
a platform (100, 140, 150) for the growth of cell cultures, said platform comprising:
a first group of non-collinearly arranged barbed fittings (120, 122, 124) at a first end of said platform (100, 140, 150);
a second group of barbed fittings (120, 122, 124) at a second end opposite said first end;
wherein said first and second groups of barbed fittings (120, 122, 124) are capable of establishing fluidic connections between said platform (100, 140, 150) and external devices and/or other fluidic systems;
a cell well (106) and/or removeable window plate (136) located adjacent said second group of barbed fittings (120, 122, 124); and
a transparent bottom substrate (e.g., 112) for imaging;
synthetic cells (e.g., 102, 130, 134, 195) mimicking biochemical materials or processes.

8. The microfluidic system for culturing modular, biomimetic compositions of claim 7 wherein the synthetic cells (e.g., 102, 128, 132, 195) are spaced and/or geometrically arranged to mimic or create a cell pairing.

9. The microfluidic system for culturing modular, biomimetic compositions of claim 8 wherein the synthetic cells (e.g., 102, 128, 132, 195) are chondrocytes (102) that model, either independently or in co-culture, a superficial zone (55C), a middle zone (55B), and a deep zone (55A) of articular cartilage for both well geometry and nanomaterial arrangement.

10. The microfluidic system for culturing modular, biomimetic compositions of claim 9 wherein the chondrocytes (102) are configured to maintain their spheroidal morphology for a time period of at least twenty-eight days.

11. The microfluidic system for culturing modular, biomimetic compositions of claim 10 wherein expression levels of phenotypic marker proteins in the chondrocytes (102) seeded in the cell well (106) are at least fifty percent greater than for chondrocytes (102) seeded in monolayer on tissue culture-treated polystyrene culture dishes; and wherein the phenotypic marker proteins are selected form the group consisting of collagen II, aggrecan, Sox-9 (SRY-Box Transcription Factor 9), and decorin.

12. The microfluidic system for culturing modular, biomimetic compositions of claim 10 wherein expression levels of de-differentiation marker proteins are at least fifty percent lower than for chondrocytes (102) seeded in monolayer on tissue culture-treated polystyrene culture dishes; and wherein the de-differentiation marker proteins are selected from the group consisting of Collagen I, Collagen X, Ki-67, and decorin.

13. The microfluidic system for culturing modular, biomimetic compositions of claim 7 wherein the synthetic cells (e.g., 102, 128, 132, 195) are mesenchymal stem cells (130), adipose cells (195), or immune cells.

14. A modular, biomimetic composition comprising:
a natural hydrogel (104) micropatterned with a plurality of wells formed using the microfluidic system for culturing modular, biomimetic compositions of claim 1.

15. The modular, biomimetic composition of claim 14, wherein the natural hydrogel (104) is an agarose hydrogel.

16. The modular, biomimetic composition of claim 14, wherein the well surface is functionalized with polydopamine (“PDA”).

17. The modular, biomimetic composition of claim 14, wherein the modular, biomimetic composition is thin film.

18. The modular, biomimetic composition of claim 14, wherein the hydrogel (104) comprises a nanofibers and/or nanoparticles (108) embedded within the hydrogel (104).

19. The modular, biomimetic composition of claim 18, wherein the nanofiber (108) comprises a polyvinyl alcohol, collagen, chitin, or a combination thereof.

20. The modular, biomimetic composition of claim 14, wherein the cell well (106) has an average diameter of from about 5 μm to about 50 μm; and wherein the cell well (106) is separated by an inter-well spacing of from about 0.1 μm to about 30 μm.
